(19) United States
(12) Patent Application Publication
(10) Pub. No.: US 2026/0137901 A1
(43) Pub. Date: May 21, 2026

(54) FILTRATION DEVICES, AND SYSTEMS AND METHODS FOR USING THEM

(71) Applicant: FILTRO MEDICAL INC., San Jose, CA (US)

(72) Inventors: Albert K. Chin, Palo Alto, CA (US); Timothy W. Robinson, Sandown, NH (US); Alexander R. Bonin, Woonsocket, RI (US)

(21) Appl. No.: 19/402,373

(22) Filed: Nov. 26, 2025

Related U.S. Application Data
(63) Continuation of application No. PCT/US2024/031506, filed on May 29, 2024.
(60) Provisional application No. 63/469,511, filed on May 29, 2023.

(51) Int. Cl. A61M 25/00

(52) U.S. Cl. CPC A61M 25/0068; A61M 25/0097; A61M 2025/0004; A61M 2202/0014; A61M 2202/0028; A61M 2202/0057; A61M 2205/0238; A61M 2205/75; A61M 2210/1071

(57) ABSTRACT

Filtration devices are provided that include a tubular outer member, an inner member slidably disposed within a lumen of the outer member such that a distal end of the inner member extends distally from the outer member, and a plurality of tubular porous members comprising first ends attached to the outer member and second ends attached to the distal end of the inner member such that axial movement of the inner member relative to the outer member axially compresses or extends the porous members to expand and collapse the porous members. Beads or other filtration media are provided within the porous members configured to adsorb or bind one or more agents from fluid passing through the porous members.

### RELATED APPLICATION DATA

The present application is a continuation of co-pending International Application No. PCT/US 2024/031506, filed May 28, 2024, which claims benefit of U.S. provisional application Ser. No. 63/469,511, filed May 29, 2023, the entire disclosures of which are expressly incorporated by reference herein.

TECHNICAL FIELD The present application relates generally to medical devices, and, more particularly, to filtration devices for filtering one or more agents in blood flowing within a subject's vasculature, e.g., for filtering one or more chemotherapy and/or other therapeutic agents delivered into a target treatment location to reduce systemic exposure, and to systems and methods for using such devices.

### BACKGROUND

Cancer is currently the second leading cause of death in the United States and is on course to exceed cardiovascular disease as the leading cause of death in the next decade. Intravenous chemotherapy is a commonly used procedures to treat a variety of cancers. However, such chemotherapy may be dose-limited by systemic toxicity of the chemotherapeutic agents delivered into a target treatment location. For example, doxorubicin (“Dox”) is a commonly used chemotherapeutic agent, whose toxicities include bone marrow suppression, gastrointestinal damage, and perhaps most notoriously irreversible cardiac failure.

In order to limit these systemic toxicities and also further increase chemotherapeutic dose to cancers being treated, a chemotherapeutic agent such as Dox may be administered intraarterially directly into vessels feeding a tumor, e.g., within a liver or other organ. However, large percentages of the chemotherapeutic agent may pass through the tumor into the patient's systemic venous circulation, thereby exposing other regions of the patient's body to the agent.

Accordingly, devices that minimize systemic exposure of chemotherapy or other therapeutic agents would be useful.

### SUMMARY

The present application is directed to generally to medical devices, and, more particularly, to filtration devices for filtering one or more agents in blood flowing within a subject's vasculature, and to systems and methods for using such devices. For example, the devices herein may be particularly useful for filtering one or more chemotherapy and/or other therapeutic agents delivered into a target treatment location, e.g., into vessels communicating with a tumor within a liver or other organ, to reduce systemic exposure.

In accordance with one example, a filtration device is provided that includes a tubular outer member comprising a proximal portion, a distal portion sized for introduction into a body lumen, and a first lumen extending between the proximal portion and an outlet in the distal portion; an elongate inner member slidably disposed within the first lumen such that a distal end of the inner member extends from the outlet and a proximal end is disposed adjacent the proximal portion; a plurality of tubular porous members comprising first ends attached to the distal portion of the outer member and second ends attached to the distal end of the inner member such that axial movement of the inner member relative to the outer member axially compresses or extends the porous members to expand and collapse the porous members; and filtration media within the porous members configured to adsorb or bind one or more agents from fluid passing through the porous members.

In accordance with another example, a filtration device is provided that includes a tubular outer member comprising a proximal portion, a distal portion sized for introduction into a body lumen, and a first lumen extending between the proximal portion and an outlet in the distal portion; an elongate inner member slidably disposed within the first lumen such that a distal end of the inner member extends from the outlet and a proximal end is disposed adjacent the proximal portion; a tubular porous member comprising a first end attached to the distal portion of the outer member and a second end attached to the distal end of the inner member such that axial movement of the inner member relative to the outer member axially compresses or extends the porous members to expand and collapse the porous member, the porous member divided into a plurality of segments between the first and second ends; and filtration media within interiors of the segments of the porous member configured to adsorb or bind one or more agents from fluid passing through the porous member.

In accordance with still another example, a method is provided for filtering one or more agents introduced into a target location that includes introducing a filtration member into a body lumen downstream of the target location, the filtration member comprising a plurality of segments of porous material spaced apart axially from one another; and expanding the segments to cause blood flowing through the body lumen to pass through the porous material to expose the blood to filtration media within the segments to adsorb or bind the one or more agents to the filtration media.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 is a cross-sectional view of a liver showing a pair of filtration devices positioned within hepatic veins to filter chemotherapy agents delivered into the liver to treat a tumor.

FIG. 2 shows an example of a filtration device including a shaft carrying a filtration member including a tubular mesh having four axial segments containing filtration media.

FIGS. 3A and 3B are details of the filtration member of FIG. 2 in collapsed and expanded configurations, respectively.

FIGS. 4A and 4B show another example of a filtration member for a filtration device in collapsed and expanded configurations, respectively.

FIG. 4C is a cross-section of the expanded filtration member of FIG. 4B taken at 4C-4C.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

In some aspects of the present disclosure, in vivo positionable filtration devices are provided that filter one or more therapeutic and/or diagnostic agents in blood flowing in a blood vessel. The filtration devices generally include a catheter or other elongate member and a filtration member coupled to the elongate member dimensioned for positioning within a blood vessel of a human or non-human subject. The filtration member includes one or more tubular porous members containing filtration media configured to filter the one or more therapeutic agents from blood.

The filtration media may include material that filters one or more therapeutic agents from the blood. For example, the therapeutic agents may include chemotherapeutic agents, e.g., Dox, and/or or non-chemotherapeutic agents.

It should be appreciated that the term “therapeutic agent” is used broadly herein and may include therapeutic particles. Furthermore, references to “filtering of a therapeutic agent” are used broadly herein and encompass the filtering of therapeutic particles. For example, particles may include free chemotherapy (or non-chemotherapy) molecules, or chemotherapy (or non-chemotherapy) loaded molecules (e.g., chemotherapy molecules that are bound to particles such as drug eluting resins or drug eluting activated carbon). Exemplary therapeutic agents may include chemotherapy agents; vasoactive agents, e.g., verapamil, nicardipine, or milrinone; Sodium tetradecyl sulfate (Sotradecol, Angiodynamics or BioNiche Pharmaceuticals); bleomycin; X-ray or MRI contrast agents; antibiotics; lytic agents (for example, and clot dissolving drugs such as tPA). In certain examples, the particles may include bland particles. Particles may include, for example, particles that occlude blood vessels of cancerous or otherwise diseased tissue. In some instances, particles may include polymers, glues, resins, activated carbon, or glass. In certain examples, the particles may be bound to radiation emitting isotopes, such as radiotherapeutic particles.

The filtration media may include material that has properties that adsorb, bind, trap, or inactivate or degrade one or more therapeutic agents. For instance, in certain examples, the filtration media may include beads or other particles that adsorb, bind, trap, inactivate, and/or degrade the therapeutic agent. Additional information related to filtration materials that may be included in the filtration devices described herein may be found in U.S. Pat. Nos. 11,406,485 and 11,554,003, the entire disclosures of which are expressly incorporated by reference herein.

As described elsewhere herein, in certain examples, the filtration media may filter a therapeutic agent from blood by adsorbing or binding to the therapeutic agent(s) to enable removal from the blood. For example, the filtration media may possess properties that adsorb a therapeutic agent, chemically bind to a therapeutic agent, and/or magnetically bind to a magnetic carrier of the therapeutic agent, without significant binding or filtration of endogenous entities in the blood. The binding between the filtration media and the therapeutic agent may be irreversible or weakly reversible. In this way, the therapeutic agent may be collected by the filtration media and removed from the blood, e.g., when the filtration device is removed.

In one example, the filtration media includes a resin that has properties that adsorbs a therapeutic agent, chemically binds to a therapeutic agent, and/or magnetically binds to a magnetic carrier bound to a therapeutic agent. Exemplary filtration materials including resins having properties to adsorb and/or chemically bind to a therapeutic agent, such as doxorubicin, may include strong acid cation exchange polymer resins; ion exchange resins; polymeric adsorbent resins without ion exchange; resins including sulfonate groups that ionically bond to the therapeutic agent; chromatography based resins, acrylic-based resins including those composed of polyacrylamide, polyacrylic acid, sodium acrylate and even vinyl copolymers, or any combination thereof. Optionally, such resins may be incorporated onto or into the porous membrane or other materials of the filtration member, e.g., formed from polymers or cloth, with examples including Nafion (Dupont), Neosepta, CMI-7000 (Membranes International), and IONAC membranes (Sybron Chemicals).

In one example, strong acid cation exchange polymer resins may be used for a mildly positively charged drug such as Dox. Other examples of compounds that chemically or physically (via adsorption) bind to a therapeutic agent such as doxorubicin, may include calsequestrin; cyclic oligosaccharides, such as cyclodextrins, including gamma-cyclodextrin; hNopp140; antibodies that specifically bind the agent, such as an anti-doxorubicin monoclonal antibody (MAD 11); nucleolar phosphoprotein; Clostridium botulinum neurotoxin B; cell membrane lipids such as cardiolipin, phophatidylserine, and phosphatic acid; nucleic acid ligands so called ‘aptamers’ including RNA and DNA; albumin; and hemoglobin.

In other examples, various other types of ion exchange resins may be used, including weak-acid cation exchange, weak-base anion exchange, strong-base anion exchange, and the like. For instance, in one example, for a negatively charged drug (e.g., heparin), a strong-base anion exchange resin may be used.

Factors such as resin functional group and porosity/cross-linking, solution temperature, pH, concentration, and ionic strength may factor into the effectiveness of the resin to bind to the therapeutic agent. In some instances, for example, cyanogen bromide activation of resins may be used to attach functional groups. In certain instances, a low cross-linked version of these resins may be used, such as 3% or less, including 2% or less. Alternatively, higher cross-linked versions may be used, if desired.

In certain examples, the filtration media may include carbon such as activated carbon (e.g., charcoal or activated charcoal) that binds to the therapeutic agent. The effectiveness of the activated carbon may vary based on factors such as pore size, shape, surface area, ash content, and hardness, for example. In some instances, the carbon may be coated with additional resin material. Furthermore, carbons and resins are inexpensive and small amounts may be used and still be effective. While larger amounts may be implemented in some instances, example amounts of carbons and resin, such as ten grams or less, such as five grams or less, and including one gram or less, may be implemented and be effective.

Exemplary resins for the filtration devices herein may include one or more of HepaSphere, QuadraSphere, Dowex 50W-X2; Dowex 50W-X4; Dowex 50W-X8; Biorad AG50W-X2; Biorad AG50W-X4; Biorad AG50W-X8; GE Sepharose Big Beads; Amberlite XAD-2; Tosoh Toyopearl MegaCap II; Purolite PAD 600; and Purolite CGC100X2. Exemplary carbons for the filtration devices may include one or more of Norit C Gran; Calgon TOG NDS 20.times.50; and QUO-YC- 1041.

In certain examples, the filtration media may include material that improves biocompatibility, such as polymethyl-methacrylate (PMMA), chitosan, heparin, etc. In some instances, for example, the resin or carbon of the filtration media may be coated or otherwise impregnated with the PMMA, chitosan, and/or heparin. Exemplary coating methods may be found in U.S. Publication No. 2010/0316694, the entire disclosure of which is expressly incorporated by reference herein.

In certain examples, the filtration media may filter a therapeutic agent from the blood by inactivating or otherwise degrading the therapeutic agent or the toxicity of the therapeutic agent. For example, the filtration media may include a catalytic material, such as an immobilized (covalently or non-covalently) enzyme that, for example, enzymatically degrades the therapeutic agent to reduce its toxicity level. Enzymatic degradation and inactivation of Dox, for example, may occur via cleavage of its sugar backbone with glycosidases contained in the liver.

In other examples, the therapeutic agent administered to the patient may be pretreated by covalently or non-covalently associating the compound with a magnetic particle, such as a magnetic nanoparticle. Accordingly, the filtration media may include magnetic material so that following treatment the magnetically bound therapeutic particle may be attracted to the magnetic material of the filtration media.

In yet other examples, the filtration media may include a basic mechanical sieve-like filter that traps a wide range of particles that are commonly used to embolize tumors, such as resin based particles, e.g., DC Beads and LC Beads (ion-exchange resins), QuadraSpheres and HepaSpheres (sodium acrylate and vinyl copolymer resin), EmboSpheres (tris-acryl resins), Bead Block and Cotonour Beads (polyvinyl alcohol resins), Onyx (ethylene vinyl copolymer, EVOH), TruFill or Histacryl (n-butyl-cyanoacrylate (nBCA) compounds), embolization coils, or activated carbon particles. Such particles may or may not be loaded with a therapeutic agent that would elute in the tumor. In such examples, the filtration media may trap these particles out of the exiting venous blood stream to prevent them from depositing in non-target organs. Moreover, the filtration media may also include a chemical based binding filtration mechanism to filter out free drug from the blood as well.

The reduction of the toxicity level may vary based on the selected filtration media, therapeutic agent, and specific filtration device configuration. In certain examples, the reduction of toxicity level may range from 50% or greater, 75% or greater, or 90% or greater.

In certain examples, the filtration member may include one or more porous membranes, e.g., containing resin beads or other filtration media within the interior of the membrane(s). For example, the filtration media may include a plurality of beads loose within the interior of the porous membrane(s), e.g., such that the beads are free to move within the interior when the filtration member is deflected and/or deformed, e.g., expanded and collapsed, such that the beads do not inhibit movement and/or manipulation of the filtration member. The porosity of the membrane(s) may vary but should be sufficient to enable blood to pass therethrough while preventing the beads or other filtration media from escaping from the membrane(s). In one example, the porosity of the membrane(s) may be selected based on the therapeutic agent particle size, e.g., to enhance capturing the therapeutic particles. Exemplary pore sizes may include pore sizes as small as forty microns to as large as three hundred microns, although other pore sizes may also be implemented, if desired.

In certain examples, the porous membrane(s) may be configured to permit blood to pass through openings in the membrane(s) and encounter the filtration media. The membrane(s) may be made from a variety of materials but should enable blood to pass through, such as fabrics, plastics, polymers, silicone, metals, metal alloys, and the like.

In one example, the membrane(s) may be formed as tubular porous members from a plurality of fibers braided into a tubular mesh structure configured to contain the filtration media therein. The number of fibers may be sufficient to provide openings smaller than the filtration media, e.g., while maintaining flexibility of the filtration member to facilitate introduction through tortuous anatomy. The resulting tubular mesh structure may be sufficiently flexible to allow the filtration membrane to be introduced into a patient's body, e.g., into tortuous anatomy within the patient's vasculature, and to facilitate directing the filtration member between collapsed and expanded configurations, e.g., by axially extending and compressing opposite ends of the mesh structure, as described further elsewhere herein.

Optionally, the material of the tubular mesh structure may be biased to the expanded or collapsed configurations, e.g., by setting a shape memory into the fiber materials, while allowing the structure to be expanded and collapsed during use. In one particular example, the tubular mesh structure may be formed from a plurality of axially inelastic fibers, e.g., formed from metal, such as Nitinol, plastic, such as polyester, and/or composite materials. In various examples, the tubular mesh structure may include between about eight and two hundred eighty eight (8-288) fibers, between about twenty four and one hundred forty four (24-144) fibers, or about sixty four (64) fibers.

Optionally, the membrane materials may themselves include material, e.g., coatings and the like, that have properties to adsorb, bind, trap, inactivate or degrade therapeutic agent(s). For instance, the membrane material may be formed from, coated with, and/or impregnated with resin, carbon, or other materials similar to those described elsewhere with respect to the filtration media.

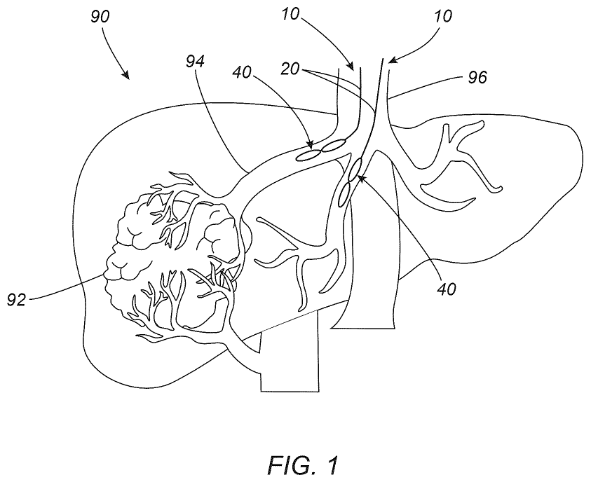

Turning to the drawings, FIG. 1 shows an example of a liver 90 of a patient including a tumor 92 therein. Generally, a filtration device 10 may be introduced into the patient's vasculature to position a porous filtration member 40 into a location to filter one or more therapeutic agents delivered into the liver 90 to treat the tumor 92. For example, as shown, two filtration devices 10 have been introduced via the interior vena cava 96 to position the filtration members 40 in respective hepatic veins 94, e.g., to filter therapeutic agent(s) in blood flowing through the hepatic veins 94 from the liver 90. Although the filtration devices described herein may have particular application in filtering agents delivered into a liver, it will be appreciated that the filtration devices herein may be introduced into other organs or locations within a patient's vasculature downstream from target treatment locations to filter agent(s) delivered into the treatment locations, and thereby minimize systemic exposure of the patient to the agent(s).

In certain examples, the filtration device(s) 10 may include a catheter 20 with a filtration member 40 disposed at a distal end 24 of the catheter 20, as shown in FIG. 1. The catheter 20 may be constructed in a variety of diameters that would fit the various sizes of blood vessels. For example, for smaller veins, such as renal or hepatic veins, the catheter 20 may include an outer diameter between about eight to fourteen millimeters (8-14 mm), while for larger veins, such as vena cave, the outer diameter may be between about twenty to thirty millimeters (20-30 mm).

Optionally, any of the filtration devices herein may be included in a system including one or more additional components, such as one or more sheaths, guidewires, and the like (not shown). For example, the systems herein may include a delivery sheath sized to receive the filtration devices to enable endovascular delivery and retrieval of the filtration devices.

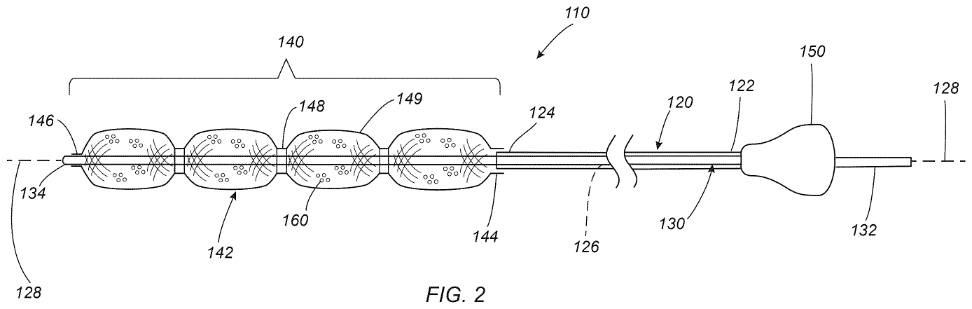

Turning to FIG. 2, an example of a filtration device 110 is shown that includes a catheter or other tubular outer member 120, an elongate inner member 130, and a filtration member 140. The outer member 120 includes a proximal portion or end 122, a distal portion or end 124 sized for introduction into a body lumen, and a first lumen 126 extending between the proximal and distal portions 122, 124, thereby defining a longitudinal axis 128. The outer member 120 may have a substantially uniform construction between the proximal and distal ends 122, 124. Alternatively, the construction may vary along the length of the outer member 120 to provide desired properties, e.g., between proximal, intermediate, and distal portions. For example, the outer member 120 may include a proximal portion adjacent the proximal end 122 that may be substantially rigid or semi-rigid, e.g., providing sufficient column strength to allow the distal end 124 (and filtration member 140 thereon) to be pushed or otherwise manipulated from the proximal end 122, while a distal portion may be substantially flexible to accommodate bending and/or introduction into tortuous anatomy. Optionally, the outer member 120 may include one or more reinforcement members, e.g., a plurality of reinforcement fibers (not shown), e.g., a plurality of fibers braided or helically wound around and/or embedded within a wall of the outer member 120 along a desired length, e.g., along at least a distal portion to prevent buckling or kinking when advanced through tortuous anatomy.

The inner member 130 is slidably disposed within the first lumen 126 such that a proximal end 132 of the inner member 130 extends from the proximal end 122 of the outer member 120 (or is otherwise positioned adjacent the proximal end 122), and a distal end 134 of the inner member 130 extends from an outlet 125 in the distal end 124 of the outer member 120. The inner member 130 may be a solid or hollow wire or other elongate member configured to slide freely within the lumen 126 of the outer member 120. Optionally, the inner member 130 may include a lumen (not shown) extending between the proximal and distal ends 132, 134, e.g., to receive a guidewire or other rail (not shown), to facilitate introduction of the filtration device 110. Optionally, the inner member 130 may include a rounded and/or other atraumatic distal end on the distal end 134, e.g., to prevent the distal end 134 from penetrating or otherwise damaging the wall of a body lumen within which the filtration device 110 is introduced.

The filtration member 140 includes a tubular porous member 142 including a first end 144 attached to the distal end 124 of the outer member 120 and a second end 146 attached to the distal end 134 of the inner member 130. Consequently, axial movement of the outer member 120 relative to the inner member 130 may axially compress or extend the porous member 142, e.g., to expand and collapse the porous member 142, as described further elsewhere herein. Filtration media 160 may be contained within the interior of the porous member 142, e.g., a plurality of beads configured to adsorb or bind one or more agents from fluid passing through the porous member 142, also as described further elsewhere herein.

In one example, the porous member 142 is formed from a plurality of fibers braided into a tubular mesh structure with opposite ends of the fibers defining the first and second ends 144, 146. The first and second ends 144, 146 of the porous member 142 may be permanently attached to the distal ends 124, 134, respectively, of the outer and inner members 120, 130. For example, a collar, heat shrink tubing, and the like may be positioned around and attached to the ends 144, 146 to secure them to the outer and inner members 120, 130. In addition or alternatively, the ends 144, 146 may attached by one or more of bonding with adhesive, sonic welding, fusing, and the like. In addition or alternatively, if the fibers are formed from plastic or other flowable material, the ends of the fibers may be melted or otherwise fused directly to the outer and inner members 120, 130.

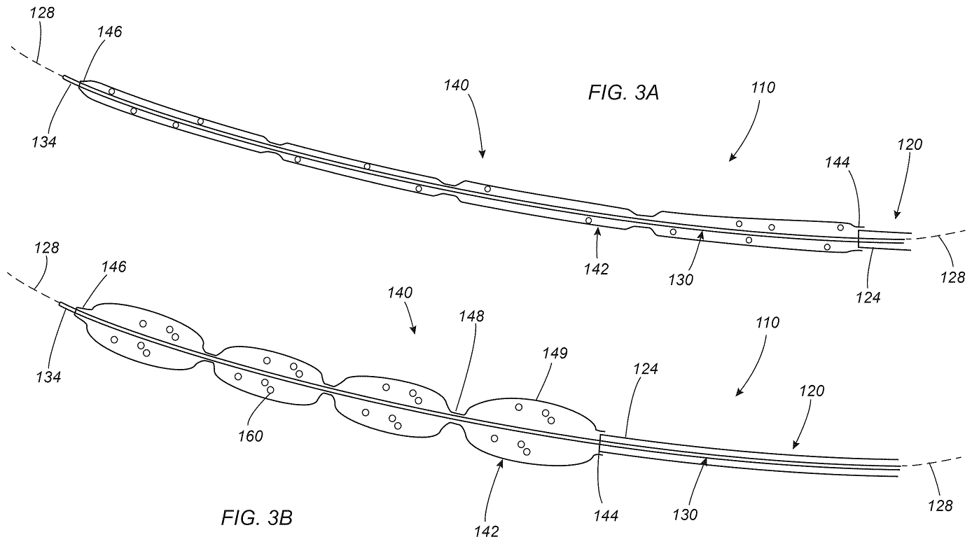

As best seen in FIGS. 3A and 3B, the porous member 142 may be divided into a plurality of segments 149 spaced apart axially between the first and second ends 144, 146. For example, the porous member 142 may include one or more waist or constricted regions 148 between the first and second ends 144, 146 that are fixed such that regions 148 cannot expand radially. In the example shown, the porous member 142 includes four segments 149 separated by three waist regions 148, although it will be appreciated that the porous member 142 may include any desired number of segments, e.g., two, three, four, five, six, or more (and corresponding numbers of one-less waist regions), if desired. In the example shown, the waist regions 148 are spaced apart substantially evenly such that the segments 149 have substantially the same length. Alternatively, the segments 149 may have one or more different lengths, if desired.

In one example, each waist region 148 may include a collar, ring, heat shrink wrap, or other material positioned around and permanently fixed to the porous member 142. In addition or alternatively, if the material of the porous member 142 is flowable, the material may be melted or otherwise modified to fuse the fibers at the waist region 148 from moving and/or adhesive or other material may be applied to fuse the fibers at the waist region 148, thereby constraining the waist region 148 from expanding. The waist region 148 may be sized to allow the underlying material of the porous member 142 to freely slide over the inner member 130 while preventing the constrained region of the porous member 142 from expanding, e.g., when the outer/inner members 120, 130 are directed proximally relative to one another.

Optionally, one or more markers (not shown) may be provided on one or more of the outer member 120, inner member 130, and/or filtration member 140. For example, a radiopaque marker may be provided on or adjacent the first and second ends 144, 146 of the porous member 142 and on each of the waist regions 148, which may facilitate monitoring the filtration member 140 during introduction and/or expansion using external imaging, such as fluoroscopy and the like. The markers may be separate rings, wires, or other radiopaque material attached to or embedded within the desired locations, and/or radiopaque material may be included in the material of the porous member 140 and/or distal ends 124, 134 of the outer and inner members 120, 130.

With further reference to FIG. 2, a hub or handle 150 may be provided on the proximal end 122 of the outer member 120, e.g., configured and/or sized for holding and/or manipulating the filtration device 110 from the proximal end 122. Optionally, one or more seals (not shown) may be provided on or within the hub 150, e.g., surrounding the inner member 130 to accommodate axial movement of the inner member 130 relative to the outer member 120 while preventing fluid within the lumen 126 from escaping out of the hub 150 and/or exterior air from entering the lumen 126. For example, as shown, the hub 150 may include a Tuohy Borst valve. Optionally, the proximal end 132 of the inner member 130 may include a hub or handle (not shown), e.g., to facilitate holding or manipulating the inner member 130 relative to the outer member 120.

Alternatively, an actuator (not shown) may be provided on the hub or handle 150 coupled to the proximal end of the inner member 130. In this alternative, the proximal end of the inner member 130 may terminate within the hub or handle 150 rather than extend proximally from the outer member 120. For example, a slider or rotating dial (not shown) on the hub 150 may be coupled to the inner member 130 that may be directed in opposite directions to direct the inner member 130 proximally or distally relative to the outer member 120.

Optionally, one or more ports (not shown) may be provided on the hub 150 (or handle). For example, a side port (not shown) may be provided on the hub 150 that communicates with the lumen 126, e.g., for delivering one or more fluids into the lumen 126 around the inner member 130. In addition, in the alternative where the inner member terminates within the hub or handle, an axial port may be provided opposite the proximal end 122 of the outer member that communicates with the lumen 126, e.g., to allow a guidewire or other instrument to pass through the hub or handle and the lumen 126. Optionally, the axial port may include one or more valves, e.g., a hemostatic valve (also not shown), which may provide a substantially fluid-tight seal, while accommodating insertion of one or more instruments into the lumen 126.

With further reference to FIGS. 3A and 3B, the outer member 120 and inner member 130 may be directed axially relative to one another to cause the filtration member 140 to selectively expand or collapse. For example, the filtration device 110 may be initially provided with the porous member 142 (and segments 149) in the collapsed configuration shown in FIG. 3A, and the outer member 120 may be directed distally relative to the inner member 130 (or conversely the inner member 130 directed proximally), thereby causing the porous member 142 to be compressed axially, thereby expanding the segments 149 radially outward to the expanded configuration shown in FIG. 3B. When desired, the outer member 120 may directed proximally, thereby axially extending the porous member 142 to direct the segments 149 back to the collapsed configuration shown in FIG. 3A.

Optionally, the outer member 120 and/or inner member 130 may include one or more stops (not shown) configured to limit axial movement of the outer and inner members 120, 130 relative to one another. For example, a distal stop may be provided that limits distal movement of the outer member 120 relative to the inner member 130 to prevent excess axial compression of the porous member 142. Similarly, a proximal stop may be provided that limits proximal movement of the outer member 120 such axial extension of the porous member 142 is limited to prevent excess tension on the fibers of the porous member 142.

During use, similar to the device 10 shown in FIG. 1, the filtration device 110 may be used to filter one or more therapeutic agents within a body lumen, e.g., to filter chemotherapy agents delivered into a liver 90 to treat tumor 92. For example, with the filtration member 140 in the collapsed configuration shown in FIG. 3A, the filtration device 110 may be introduced into a patient's vasculature and advanced to a desired position, e.g., downstream of a target treatment region. For example, similar to the device 10 shown in FIG. 1, the filtration device 110 may be manipulated to position the distal end 134 of the inner member 130, and consequently, the filtration member 140, within a hepatic vein 94 downstream from the tumor 92.

Optionally, the body lumen may be accessed using one or more additional instruments (not shown), which may be part of a system or kit including the filtration device 110, e.g., including one or more introducer sheaths, guide catheters, and/or guidewires (not shown).

For example, a guidewire or other rail (not shown) may be introduced from a percutaneous puncture, cut-down, or other access site created at a peripheral location (not shown), and the guidewire may be advanced through the patient's vasculature from the entry site, e.g., alone or with the aid of a guide catheter (not shown). For example, a distal end of a guide catheter (not shown) may be advanced over the guidewire to the desired location, and the guide catheter may then be used to the filtration device 110, e.g., over the guidewire or after removing the guidewire.

Once the distal end 134 is positioned at the desired location, the outer member 120 may be advanced distally to direct the filtration member 140 to the expanded configuration, e.g., as shown in FIG. 3B. Optionally, positioning and expansion may be monitored using external imaging, e.g., using fluoroscopy and the like to identify markers on the filtration device 110, as described elsewhere herein.

Once the filtration member 140 is properly positioned and expanded, one or more therapeutic agents may be delivered to the target treatment location, e.g., into the liver 90 shown in FIG. 1, using conventional methods. Blood (and any agents carried by the blood) passing from the treatment location, e.g., into the hepatic artery 94 shown in FIG. 1, may encounter the expanded filtration member 140, thereby entering the openings in the porous member 142 to allow the filtration media 160 to filter the agents from the blood.

After delivery of the therapeutic agents is discontinued and sufficient time has passed, the outer member 120 may be directed proximally to direct the filtration member 140 back to the collapsed configuration, whereupon the filtration device 110 may be removed and the procedure completed using conventional methods.

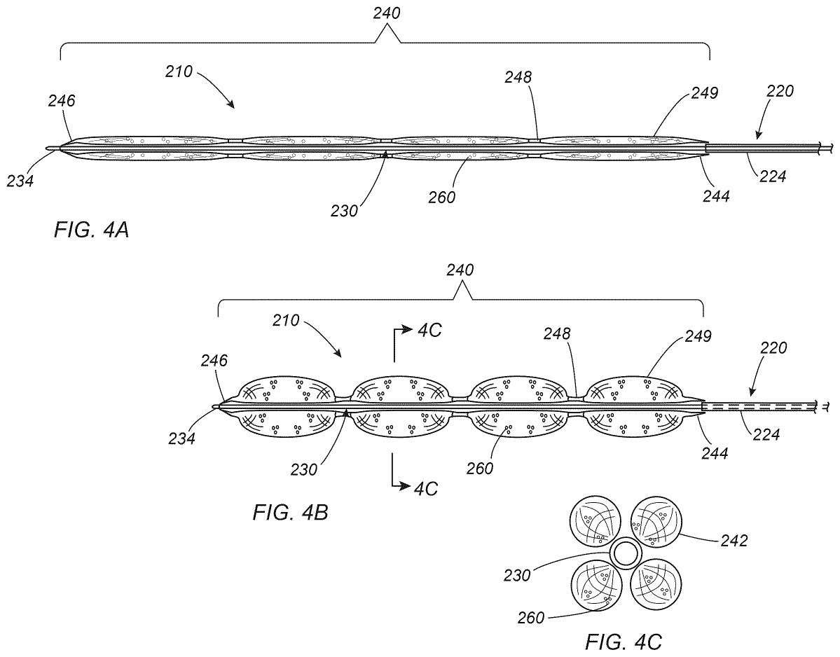

Turning to FIGS. 4A and 4B, another example of a filtration device 210 is shown that is constructed generally similar to the filtration device 110, i.e., including an outer member 220, an inner member 230, and a filtration member 240. Unlike the filtration device 110, the filtration member 240 includes a plurality of tubular porous members 242 containing filtration media 260 attached to the outer and inner members 220, 230.

As shown, each tubular porous member 242 includes a first end 244 attached to the distal end 224 of the outer member 220 and a second end 246 attached to the distal end 234 of the inner member 230. The first and second ends 244, 246 of the porous members 242 may be spaced apart from one another around the circumference of the distal ends 224, 234 of the outer and inner members 220, 230 such that the porous members 242 lie against the outer wall of the inner member 230 between the first and second ends 244, 246. Consequently, the inner member 230 remains outside the interiors of the porous members 242 and the porous members 242 are spaced apart from one another around the circumference of the inner member 230, as best seen in FIG. 4C. In one example, each porous member 242 may be formed by braiding a plurality of fibers into a tubular mesh structure, or otherwise formed to have a desired pore size, as described elsewhere herein.

In the example shown in FIG. 4C, the filtration device 210 includes four porous members 242 spaced apart substantially evenly around the inner member 230. It will be appreciated that any desired number of porous members 242 may be provided, e.g., two, three, four, five, six, or more.

In addition, each porous member 242 includes a plurality of axial segments 249 spaced apart between the first and second ends 244, 246. For example, one or more waist regions 248 may be provided along the lengths of the porous members 242, e.g., three waist region 248 as shown to provide four segments 249 for each of the porous members 242. For example, a collar, ring, heat shrink wrap, and the like may be attached around the porous members 242 to create a waist region 248 at the same location on all of the porous members 242, similar to the previous examples. In addition or alternatively, the porous members 242 may be fixed at the waist regions by bonding with adhesive, fusing, sonic welding, and the like. The waist regions 248 may allow the porous members 242 to slide over the inner member 230, e.g., such that only the first and second ends 244, 246 of the porous members 242 are fixed relative to the outer and inner members 220, 230.

Consequently, similar to the filtration device 110, axial movement of the inner member 230 relative to the outer member 220 may axially compress or extend the porous members 242 simultaneously, to expand and collapse the porous members 242. For example, as shown in FIG. 4A, the filtration device 210 may be provided with the porous members 242 in a collapsed configuration, e.g., with sufficient tension applied to the fibers or other mesh structure of the porous members 242, e.g., to maintain the porous members immediately adjacent the inner member 230 to minimize an outer cross-section of the filtration member 240. Once introduced and positioned at a desired location, the outer member 220 may be advanced distally relative to the inner member 230 (or the inner member directed proximally) to axially compress the porous members 242 and cause the segments 249 to expand radially outwardly to the expanded configuration, as shown in FIGS. 4B and 4C. After using the filtration device 210 to filter one or more therapeutic agents, the filtration member 240 may be returned to the collapsed configuration shown in FIG. 4A and removed from the patient's body, similar to the other devices described herein.

The foregoing disclosure of various examples has been presented for purposes of illustration and description. It is not intended to be exhaustive or to limit the invention to the precise forms disclosed. Many variations and modifications of the embodiments described herein will be apparent to one of ordinary skill in the art in light of the above disclosure.

Further, in describing representative embodiments, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A filtration device, comprising:
a tubular outer member comprising a proximal portion, a distal portion sized for introduction into a body lumen, and a first lumen extending between the proximal portion and an outlet in the distal portion;
an elongate inner member slidably disposed within the first lumen such that a distal end of the inner member extends from the outlet and a proximal end is disposed adjacent the proximal portion;
a plurality of tubular porous members comprising first ends attached to the distal portion of the outer member and second ends attached to the distal end of the inner member such that axial movement of the inner member relative to the outer member axially compresses or extends the porous members to expand and collapse the porous members; and
filtration media within the porous members configured to adsorb or bind one or more agents from fluid passing through the porous members.

2. The filtration device of claim 1, wherein the porous members comprise a plurality of segments axially spaced apart from one another between the first and second ends.

3. The filtration device of claim 2, further comprising one or more waist regions dividing the porous members into the plurality of segments.

4. The filtration device of claim 3, wherein the one or more waist regions are slidable over the inner member such that the porous members are free to slide over the inner member between the first and second ends.

5. The filtration device of claim 2, wherein the one or more waist regions comprise a single waist region dividing the porous members into two segments between the first and second ends.

6. The filtration device of claim 2, wherein the one or more collars comprise two waist segments dividing the porous members into three segments between the first and second ends.

7. The filtration device of claim 2, wherein the one or more collars comprise three waist regions dividing the porous members into four segments between the first and second ends.

8. The filtration device of claim 1, wherein each of the porous members comprise a plurality of fibers braided together between the first and second ends.

9. (canceled)

10. The filtration device of claim 8, wherein fibers are braided to define openings that are smaller than a cross-section of the filtration media contained within the porous members.

11. The filtration device of claim 1, wherein the porous members comprise mesh defining openings that are smaller than a cross-section of the filtration media contained within the porous members.

12. The filtration device of claim 1, wherein the inner member comprises a second lumen extending between the proximal end and an outlet in the distal end.

13. The filtration device of claim 1, further comprising a hub on the proximal portion of the outer member and wherein the proximal end of the inner member extends proximally from the hub such that the inner member is slidable axially relative to the hub.

14. The filtration device of claim 13, further comprising a seal within the hub at least partially surrounding the inner member.

15. The filtration device of claim 1, further comprising a handle on the proximal portion of the outer member and an actuator on the handle coupled to the proximal end of the inner member such that movement of the actuator causes the inner member to move axially relative to the outer member.

16. The filtration device of claim 1, wherein the filtration media comprise resin beads.

17. The filtration device of claim 16, wherein the resin beads are configured to adsorb or chemically bind one or more chemotherapeutic agents.

18. The filtration device of claim 16, wherein the resin beads comprise one or more of an ion exchange resin, a strong acid cation polymer resin, a polymeric adsorbent resin without ion exchange, and one or more sulfonate groups that ionically bind to the one or more chemotherapeutic agents.

19. The filtration device of claim 16, wherein the resin beads are coated with one or more of heparin, polymethyl-methacrylate, and chitosan.

20. A filtration device, comprising:
a tubular outer member comprising a proximal portion, a distal portion sized for introduction into a body lumen, and a first lumen extending between the proximal portion and an outlet in the distal portion;
an elongate inner member slidably disposed within the first lumen such that a distal end of the inner member extends from the outlet and a proximal end is disposed adjacent the proximal portion;
a plurality of tubular porous members comprising first ends attached to the distal portion of the outer member and second ends attached to the distal end of the inner member and a plurality of segments spaced apart axially between the first and second ends such that axial movement of the inner member relative to the outer member axially compresses or extends the porous members to expand and collapse the segments; and
filtration media within the segments of the porous members configured to adsorb or bind one or more agents from fluid passing through the porous members.

21-40. (canceled)

41. A method for filtering one or more agents introduced into a target location, comprising:
introducing a filtration member into a body lumen downstream of the target location, the filtration member comprising a plurality of segments of porous material spaced apart axially from one another;
expanding the segments to cause blood flowing through the body lumen to pass through the porous material to expose the blood to filtration media within the segments to adsorb or bind the one or more agents to the filtration media.

42-45. (canceled)
